(19) United States
(12) Patent Application Publication
(10) Pub. No.: US 2022/0388375 A1
(43) Pub. Date: Dec. 8, 2022

(54) Air Sterilization Assembly

(71) Applicants: Mark Downey, Jeffersontown, KY (US); Rishu Sharma, Jeffersontown, KY (US)

(72) Inventors: Mark Downey, Jeffersontown, KY (US); Rishu Sharma, Jeffersontown, KY (US)

(21) Appl. No.: 17/340,721

(22) Filed: Jun. 7, 2021

(51) Int. Cl. B60H 3/00; A61L 9/20

(52) U.S. Cl. CPC B60H 3/0078; A61L 9/20; A61L 2209/12; A61L 2209/15; A61L 2209/16

(57) ABSTRACT

An air sterilization assembly for passenger compartment air sterilization includes a substrate and a set of bulbs. The substrate is mountable within an air conduit of a passenger compartment ventilation system of a vehicle so that the substrate is mounted to the air conduit without substantially reducing airflow through the air conduit. The set of bulbs is engaged to the substrate and is operationally engageable to an electrical circuit of the vehicle. The bulbs emit ultraviolet C radiation, which neutralizes pathogens in air passing through the air conduit.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sterilization assemblies and more particularly pertains to a new sterilization assembly for passenger compartment air sterilization. The present invention discloses a frame to which a set of bulbs is engaged. The frame is configured to be removably engageable to a return filter of a passenger compartment ventilation system. The bulbs emit ultraviolet C radiation to neutralize pathogens in air circulated by the passenger compartment ventilation system.

### (2) Description of Related Art Including Information Disclosed Under 37 CFR 1.97 and 1.98

The prior art relates to sterilization assemblies and in particular sterilization assemblies for neutralizing pathogens in air recirculated in passenger compartments of vehicles. Such prior art sterilization assemblies are not configured to be removably engageable to a return filter of a passenger compartment ventilation system.

### BRIEF SUMMARY OF THE INVENTION

An embodiment of the disclosure meets the needs presented above by generally comprising a substrate and a set of bulbs. The substrate is configured to be mountable within an air conduit of a passenger compartment ventilation system of a vehicle so that the substrate is mounted to the air conduit without substantially reducing airflow through the air conduit. The set of bulbs is engaged to the substrate and is configured to operationally engage an electrical circuit of the vehicle. The bulbs are configured to emit ultraviolet C radiation and thus are configured to neutralize pathogens in air passing through the air conduit.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of an air sterilization assembly according to an embodiment of the disclosure.

FIG. 2 is an isometric perspective view of an embodiment of the disclosure.

FIG. 3 is an in-use view of an embodiment of the disclosure.

FIG. 4 is an in-use view of an embodiment of the disclosure.

FIG. 5 is a block diagram of an embodiment of the disclosure.

### DETAILED DESCRIPTION OF THE INVENTION

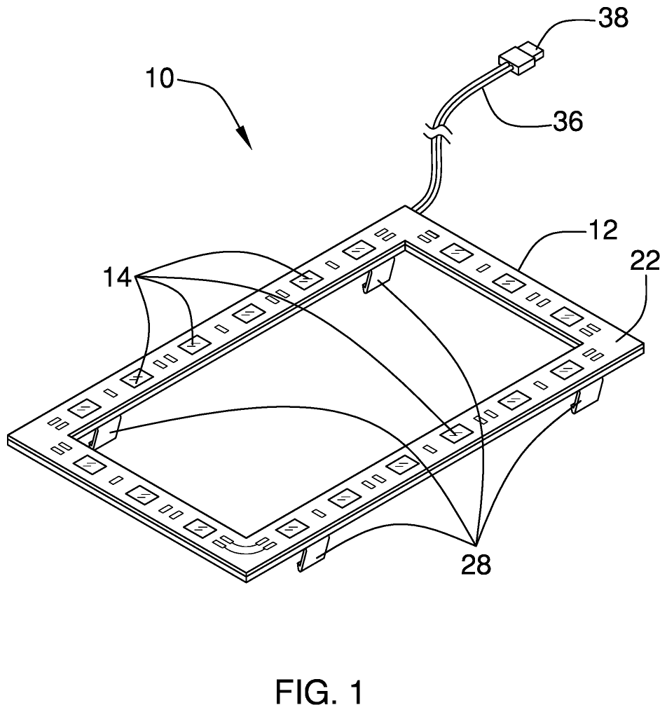

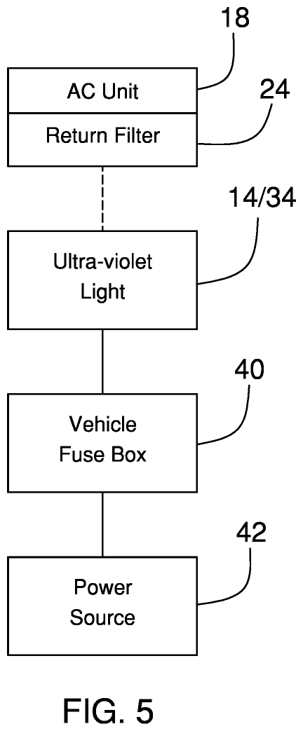

With reference now to the drawings, and in particular to FIGS. 1 through 5 thereof, a new sterilization assembly embodying the principles and concepts of an embodiment of the disclosure and generally designated by the reference numeral 10 will be described.

As best illustrated in FIGS. 1 through 5, the air sterilization assembly 10 generally comprises a substrate 12 and a set of bulbs 14. The substrate 12 is configured to be mountable within an air conduit 16 of a passenger compartment ventilation system 18 of a vehicle 20 so that the substrate is mounted to the air conduit 16 without substantially reducing airflow through the air conduit 16.

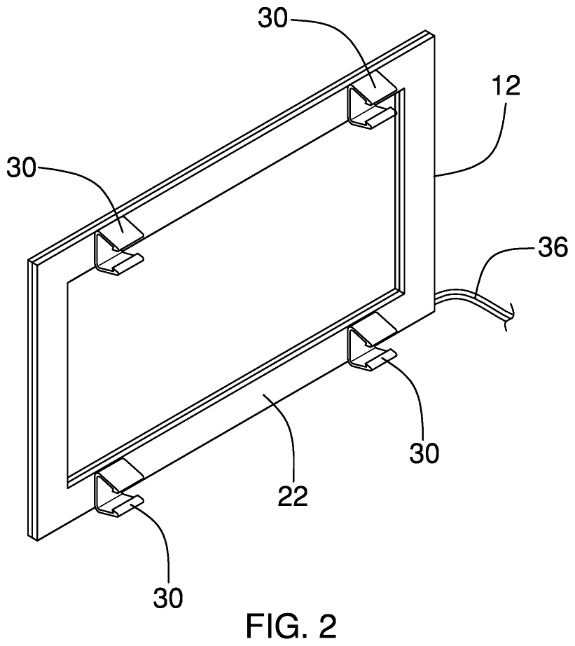

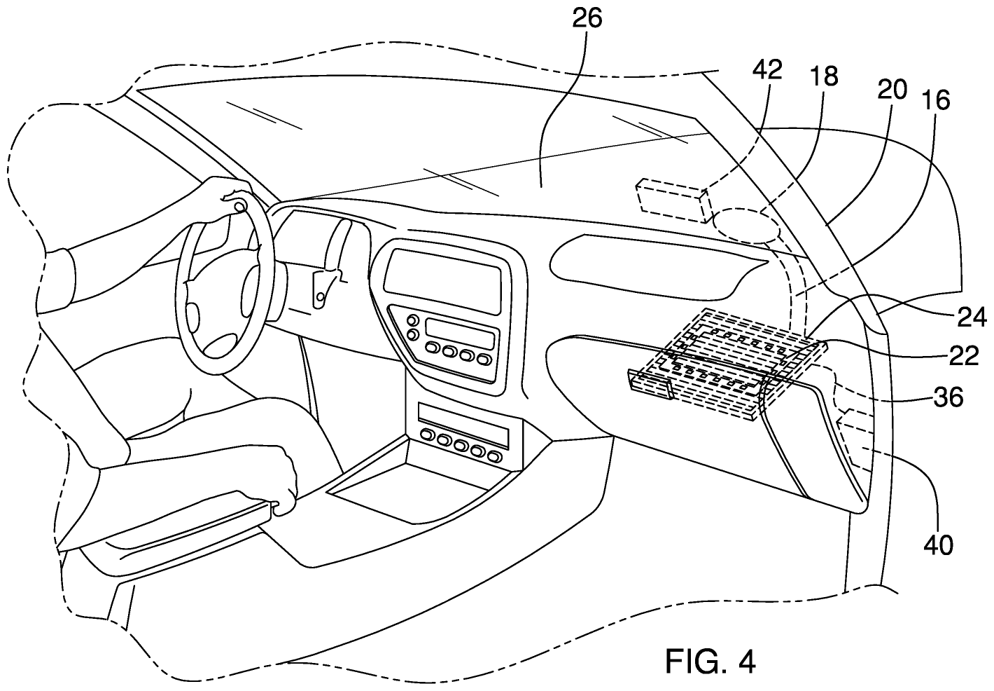

The substrate 12 may comprise a frame 22, or be of an alternative configuration, such as, but not limited to, panels, tubes, and the like. The frame 22 may be shaped complementarily to a return filter 24, as shown in FIG. 2, which is removably engageable to the air conduit 16, as shown in FIG. 4. In such case, the air conduit 16 is configured to allow intake of air through the return filter 24 and into the passenger compartment ventilation system 18 from a passenger compartment 26 of the vehicle 20. The present invention also anticipates the substrate 12 being configured to be mountable to an air conduit 16 of the passenger compartment ventilation system 18 wherein the air conduit 16 is configured to allow passage of air into the passenger compartment 26.

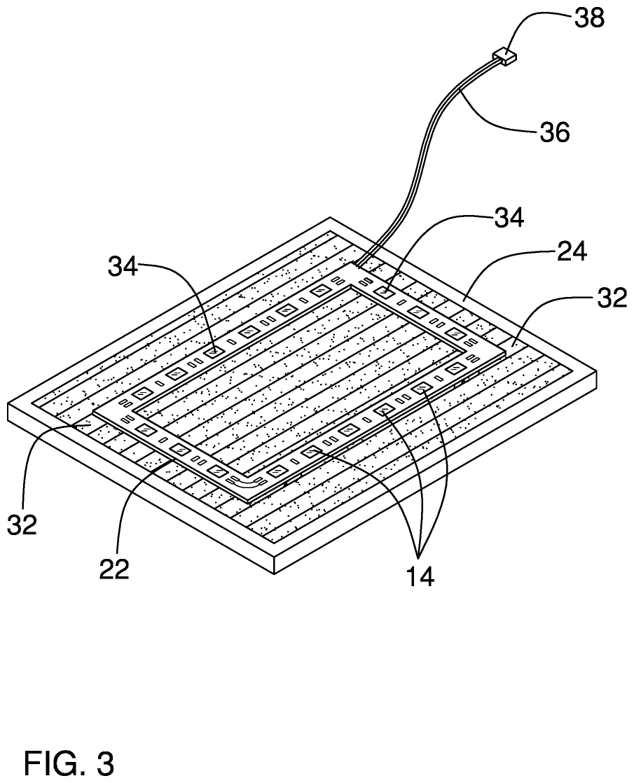

A plurality of fasteners 28 is engaged to the substrate 12 and is configured to selectively engaging the return filter 24. The fasteners 28 are configured to removably mount the substrate 12 to the return filter 24. Each fastener 28 may comprise a clip 30, which is configured to selectively engage a respective pleat 32 of the return filter 24, as shown in FIG. 3. The present invention also anticipates other fastening means for fastening the substrate 12 to the air conduit 16, such as, but not limited to, magnets, adhesives, screws, and the like.

The set of bulbs 14 is engaged to the substrate 12 and is configured to operationally engage an electrical circuit of the vehicle 20. The bulbs 14 are configured to emit ultraviolet C radiation and thus are configured to neutralize pathogens in air passing through the air conduit 16. Each bulb 14 comprises a light emitting diode 34, or other ultraviolet C radiation emitter, such as, but not limited to, xenon lamps, excimer lamps, low-pressure mercury lamps, and the like.

The present invention is anticipated to be useful in preventing circulation of pathogens through the passenger compartment ventilation system 18, thereby reducing transmission of the pathogen amongst passengers in the passenger compartment 26. In particular, vehicles 20 that carry passengers who are not of the same household, such as, but not limited to, taxis, ride share vehicles, busses, and the like, would be improved by installation of the air sterilization assembly 10.

A cord 36 is engaged to the set of bulbs 14. A connector 38 is engaged to the cord 36 distal from the set of bulbs 14. The connector 38 is configured to engage a fuse box 40 of the vehicle 20 so that the set of bulbs 14 is operationally engaged to the fuse box 40 and thus to a battery 42 of the vehicle 20. The present invention also anticipates the set of bulbs 14 being integral to the passenger compartment ventilation system 18 by incorporating the set of bulbs 14 during manufacture of the vehicle 20.

In use, the clips 30 are used to affix the frame 22 to the return filter 24 so that the light emitting diodes 34 point into the air conduit 16 when the return filter 24 is installed. The cord 36 is extended from the frame 22 to the fuse box 40 and is operationally engaged to the fuse box 40 using the connector 38 so that the battery 42 can power the light emitting diodes 34. The connector 38 can be engaged to the fuse box 40 such that the light emitting diodes 34 have power and emit ultraviolet C radiation whenever power is supplied to the passenger compartment ventilation system 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An air sterilization assembly comprising:
a substrate configured to be mountable within an air conduit of a passenger compartment ventilation system of a vehicle, such that the substrate is mounted to the air conduit without substantially reducing airflow through the air conduit; and
a set of bulbs engaged to the substrate and being configured for operationally engaging an electrical circuit of the vehicle, the bulbs being configured for emitting ultraviolet C radiation, wherein the bulbs are configured for neutralizing pathogens in air passing through the air conduit.

2. The air sterilization assembly of claim 1, wherein the substrate comprises a frame.

3. The air sterilization assembly of claim 2, wherein the frame is shaped complementarily to a return filter removably engageable to the air conduit.

4. The air sterilization assembly of claim 1, further including a plurality of fasteners engaged to the substrate and being configured for selectively engaging a return filter removably engageable to the air conduit, wherein the fasteners are configured for removably mounting the substrate to the return filter.

5. The air sterilization assembly of claim 4, wherein each fastener comprises a clip configured for selectively engaging a respective pleat of the return filter.

6. The air sterilization assembly of claim 1, wherein each bulb comprises a light emitting diode.

7. The air sterilization assembly of claim 1, further including:
a cord engaged to the set of bulbs; and
a connector engaged to the cord distal from the set of bulbs, the connector being configured for engaging a fuse box of the vehicle, such that the set of bulbs is operationally engaged to the fuse box and a battery of the vehicle.

8. An air sterilization system comprising:
a vehicle comprising a passenger compartment ventilation system;
a substrate mounted within an air conduit of the passenger compartment ventilation system, such that the substrate does not substantially reduce airflow through the air conduit; and
a set of bulbs engaged to the substrate and being operationally engaged an electrical circuit of the vehicle, the bulbs being configured for emitting ultraviolet C radiation, wherein the bulbs are configured for neutralizing pathogens in air passing through the air conduit.

9. The air sterilization system of claim 8, wherein the substrate comprises a frame.

10. The air sterilization system of claim 9, wherein the frame is shaped complementarily to a return filter removably engageable to the air conduit.

11. The air sterilization system of claim 8, further including a plurality of fasteners engaged to the substrate and being configured for selectively engaging a return filter removably engageable to the air conduit, such that the fasteners are positioned for removably mounting the substrate to the return filter.

12. The air sterilization system of claim 11, wherein each fastener comprises a clip configured for selectively engaging a respective pleat of the return filter.

13. The air sterilization system of claim 8, wherein each bulb comprises a light emitting diode.

14. The air sterilization system of claim 8, further including:
a cord engaged to the set of bulbs; and
a connector engaged to the cord distal from the set of bulbs, the connector being engaged to a fuse box of the vehicle, such that the set of bulbs is operationally engaged to the fuse box and a battery of the vehicle.

15. An air sterilization assembly comprising:
a substrate configured to be mountable within an air conduit of a passenger compartment ventilation system of a vehicle, such that the substrate is mounted to the air conduit without substantially reducing airflow through the air conduit, the substrate comprising a frame, the frame being shaped complementarily to a return filter removably engageable to the air conduit;
a plurality of fasteners engaged to the substrate and being configured for selectively engaging the return filter, wherein the fasteners are configured for removably mounting the substrate to the return filter, each fastener comprising a clip configured for selectively engaging a respective pleat of the return filter;
a set of bulbs engaged to the substrate and being configured for operationally engaging an electrical circuit of the vehicle, the bulbs being configured for emitting ultraviolet C radiation, wherein the bulbs are configured for neutralizing pathogens in air passing through the air conduit, each bulb comprising a light emitting diode;
a cord engaged to the set of bulbs; and
a connector engaged to the cord distal from the set of bulbs, the connector being configured for engaging a fuse box of the vehicle, such that the set of bulbs is operationally engaged to the fuse box and a battery of the vehicle.
